(19) United States
(12) Patent Application Publication
(10) Pub. No.: US 2020/0082935 A1
(43) Pub. Date: Mar. 12, 2020

(54) METHOD AND SYSTEM FOR MANAGEMENT OF HEALTHCARE PRACTICES

(71) Applicant: SE HEALTHCARE QUALITY CONSULTING, LLC, Charleston, SC (US)

(72) Inventors: Stephanie SARGENT, Charleston, SC (US); Rick HAMMER, Hershey, PA (US); Brian DONOVAN, Shippensburg, PA (US); Victor BOUDOLF, Charleston, SC (US); Anthony GAENZLE, Lancaster, PA (US); John YOUNGBLOOD, Charleston, SC (US); Douglas G. SHAND, Lancaster, PA (US); Chris RUMPF, Wernersville, PA (US)

(21) Appl. No.: 16/556,431

(22) Filed: Aug. 30, 2019

Related U.S. Application Data
(60) Provisional application No. 62/727,796, filed on Sep. 6, 2018.

(51) Int. Cl. G16H 40/20

(52) U.S. Cl. CPC G16H 40/20

(57) ABSTRACT

A healthcare management system and a method of: determining and enhancing a patient experience with a healthcare practice or group; determining and enhancing a clinical effectiveness with a healthcare practice or group; and enhancing reimbursement with a healthcare practice or group by developing and monitoring key drivers of a patient experience, developing and monitoring key drivers of a clinical effectiveness, and compiling results of the patient experience and results of the clinical effectiveness to determine the strengths of the practice of group.

### FIELD OF THE INVENTION

The invention is directed to healthcare industry. In particular, the invention is directed to a system and method for determining the patient experience and clinical effectiveness of a practice or group and enhancing the reimbursement the practice or group receives.

### BACKGROUND OF THE INVENTION

The patient experience of care is increasingly a driver of a practice's reputation, profitability, volume and ultimately its success. In some cases, if the healthcare organization is participating in a quality-based payment program, payor reimbursement rates are tied to patient experience performance.

With rising consumerism, potential patients can easily search the Internet and discover a wealth of publicly posted information about healthcare providers. Websites such as Healthgrades, WebMD, Physician Compare, Yelp, and Angie's List (to name a few) publish information on quality, safety and patients' ratings of healthcare providers.

Healthcare organizations who proactively measure, endeavor to improve and monitor their patient experience performance are less likely to find themselves surprised by negative online reviews—which could exist on the Internet for perpetuity.

The patient experience, defined as whether something that should happen in a health care setting (such as clear and effective communication with a provider) actually happened, is a clear marker of quality. There is an undisputed correlation between the patient experience and clinical outcomes. With an increased focus on outcomes and population health, healthcare organizations that achieve higher patient experience scores should be recognized and rewarded.

Practices with higher scores relative to their peers are more likely to retain their customer base. Taken a step further, the longer the relationship between a patient and their provider, the better the continuity of care (and necessarily quality).

There are numerous studies that emphasize the value of the patient experience. Findings include: at both the practice and individual provider levels, patient experience positively correlates to processes of care for both prevention and disease management; patients' experiences with care, particularly communication with providers, correlate with adherence to medical advice and treatment plans; patients with better care experiences often have better health outcomes; and good patient experience is associated with lower medical malpractice risk.

It is, therefore, an object of the invention to provide a system and method for determining the patient experience and clinical effectiveness of a practice or group. It is also an object to provide a system and method for enhancing the reimbursement the practice or group receives.

### SUMMARY OF THE INVENTION

An object is to provide a method and system to drive value for healthcare providers along at least the following dimensions: clinical quality, efficiency (cost and utilization), patient experience/engagement, physician health and burnout, and patient safety.

An object is to provide a method and system which reduces malpractice claims, increases customer loyalty & reputation in the market, and increases reimbursement through a clear, concise value proposition.

An embodiment is directed to a method of determining a patient experience with a healthcare practice or group by developing and monitoring key drivers of the patient experience.

An embodiment is directed to a method of determining a clinical effectiveness of a healthcare practice or group by developing and monitoring key drivers of the clinical effectiveness.

An embodiment is directed to a method of enhancing reimbursement with a healthcare practice or group by developing and monitoring key drivers of a patient experience, developing and monitoring key drivers of a clinical effectiveness, and compiling results of the patient experience and results of the clinical effectiveness to determine the strengths of the practice of group.

An embodiment is directed to a healthcare management system of enhancing a patient experience with a healthcare practice or group by developing, monitoring and enhancing key drivers of the patient experience.

An embodiment is directed to a healthcare management system of enhancing a clinical effectiveness of a healthcare practice or group by developing, monitoring and enhancing key drivers of the clinical effectiveness.

An embodiment is directed to a healthcare management system of enhancing reimbursement with a healthcare practice or group by developing and monitoring key drivers of a patient experience, developing and monitoring key drivers of a clinical effectiveness, and compiling results of the patient experience and results of the clinical effectiveness to determine the strengths of the practice of group.

An embodiment is directed to a method of determining and enhancing a patient experience with a healthcare practice or group. The method includes: collecting data from a patient on a first computer; transferring the collected data from the first computer to a second computer or server; compiling, by the second computer or server, the data into defined categories on the second computer or server; comparing, on the second computer or server the compiled data against nationally recognized benchmarks; determining and ranking key drivers on the second computer or server; and determining the healthcare practice or group performance on the second computer or server.

An embodiment is directed to a method of determining and enhancing a clinical effectiveness of a healthcare practice or group. The method includes: developing core questions to be answered by physicians or the healthcare practice or group regarding clinical effectiveness and storing the core questions on a second computer or server; developing specialty questions for various specialty areas of healthcare practice or group to be answered by the physicians or healthcare practice or group regarding clinical effectiveness and storing the specialty questions on the second computer or server; transferring the core questions and relevant specialty questions to a first computer; collecting data from physicians or healthcare practice or group on the first computer; transferring the collected data from the first computer to the second computer or server; compiling the collected data into categories on the second computer or server; calculating a clinical effectiveness score for the healthcare practice or group for each category on the second computer or server; and comparing on the second computer or server, the calculated clinical effectiveness score against national benchmarks.

An embodiment is directed to a method of determining and enhancing the reimbursement effectiveness of a healthcare practice or group. The method determining and enhancing the reimbursement effectiveness of a healthcare practice or group including: determining a patient experience with a healthcare practice or group; determining a clinical effectiveness of a healthcare practice or group; analyzing and compiling the patient experience results and the clinical effectiveness results; determining the strengths of the healthcare practice or group; and presenting the strengths of the healthcare practice or group to an insurance provider to demonstrate risk mitigation and to increase the reimbursement payment or percentage that the healthcare practice or group receives from the insurance provider. The method of determining a patient experience includes; collecting data from a patient; compiling the data into defined categories; comparing the compiled data against nationally recognized benchmarks; determining and ranking key drivers; and determining the healthcare practice or group performance. The method of determining a patient experience includes: developing core questions to be answered by physicians or the healthcare practice or group regarding clinical effectiveness; developing specialty questions for various specialty areas of healthcare practice or group to be answered by the physicians or healthcare practice or group regarding clinical effectiveness; collecting data is response to the core questions and specialty questions from physicians or healthcare practice or group; compiling the collected data into categories; calculating a clinical effectiveness score for the healthcare practice or group for each category; and comparing the calculated clinical effectiveness score against national benchmarks.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 is a flow chart illustrating the method of for determining the patient experience of the practice or group.

FIG. 2 is a flow chart illustrating the method of for determining the clinical effectiveness of the practice or group.

FIG. 3 is a flow chart illustrating the method of for determining and managing the reimbursement enhancement of the practice or group.

FIG. 4 is a diagram used to display the key drivers of the patient experience of the practice or group, with descriptors provided for each quadrant of the chart.

FIG. 5 is a diagram similar to FIG. 4, with samples key drivers provided in the quadrants.

FIG. 6 illustrates an example graphic representation of the results of the patient experiences of the practice or group compared to benchmarks.

FIG. 7 illustrates a representative gap analysis correlated with the chart used to display the key drivers.

FIG. 8 illustrates a representative comparison of the key drivers correlated with the chart used to display the key drivers with the benchmarks.

FIG. 9 illustrates a representative graph of the performance improvement opportunities for the practice or group.

FIG. 10 is an example of prioritized tips that are provided as a result of the patient experience analysis.

FIG. 11 illustrates an example graphic representation of the results of the clinical effectiveness of the practice or group compared to benchmarks.

FIG. 12 is an example of prioritized tips that are provided as a result of the clinical effectiveness analysis.

### DETAILED DESCRIPTION OF THE INVENTION

The description of illustrative embodiments according to principles of the present invention is intended to be read in connection with the accompanying drawings, which are to be considered part of the entire written description. In the description of embodiments of the invention disclosed herein, the features and benefits of the invention are illustrated by reference to the preferred embodiments. Accordingly, the invention expressly should not be limited to such preferred embodiments illustrating some possible non-limiting combination of features that may exist alone or in other combinations of features, the scope of the invention being defined by the claims appended hereto.

The system and method for management of health care practices and/or groups includes developing a system and method for determining the patient experience of the practice or group, developing a system and method for determining the clinical effectiveness of the practice or group, and developing a system and method for facilitating reimbursement enhancement of the practice or group from the insurance providers.

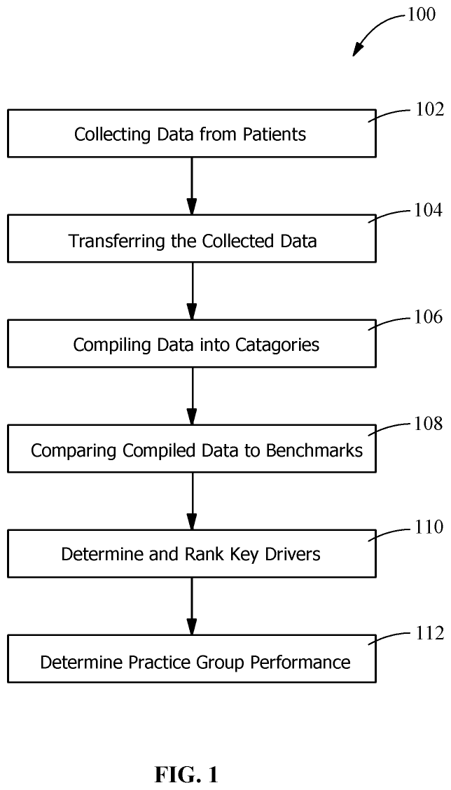

As best shown in FIG. 1, a healthcare management system 100 of determining and enhancing the patient experience and a method of determining and enhancing the patient experience with a healthcare practice or group includes the steps of: collecting data from patients 102, for example, but not limited to, on a first computer or server; transferring the collected data 104, for example, but not limited to, from the first computer or server to a second computer or server; compiling 106, for example, but not limited to, by the second computer or server, the data into meaningful categories on the second computer or server; responsive to the step of compiling, comparing 108, on the second computer or server the compiled data against nationally recognized benchmarks; responsive to the steps of compiling and comparing, determining and ranking the key drivers 110, for example, but not limited to, on the second computer or server; and responsive to the step of comparing, determining the practice or group performance 112, for example, but not limited to, on the second computer or server.

The patient experience data is gathered, compiled, analyzed and summarized. The data is compared to benchmarks, such as, but not limited to the Consumer Assessment of Healthcare Providers and Systems Comparative Database (Comparative Database) and SE Healthcare's national patient experience database (SE Healthcare Database). The Comparative Database is a repository for data from selected surveys and represents all providers in the United States who submit data to the Comparative Database. Consequently, this database provides the largest pool of standardized comparative data available. The benchmarks used from the Comparative Database are calculated by taking the mean across all respondents and response options. The SE Healthcare Database represents all practices on SE Healthcare's patient experience survey instrument. The benchmarks from the Comparative Database and the SE Healthcare Database are continually updated to ensure that the benchmarks are based on the most current data available.

The two primary patient experience compiled measures consist of the rating of provider and likelihood to recommend the practice. These two patient experience survey items are considered “global ratings” that summarize patients' overall experience and satisfaction with their provider and the practice altogether.

Consumerism is driving medical practices to become more competitive and to strive harder to retain their customer base as well as attract new customers. It is, therefore, important to understand patient loyalty. In order to do so, it is required to determine and measure the key drivers of patient satisfaction with their providers and the practice.

Using over a million individual data points which have been gathered over many years, the key drivers were developed to gain deeper insights into the patient experience with the goal of providing targeted actions for providers and the practice to improve their patient experience ratings.

The patient loyalty index is the average of the two primary patient experience compiled measures discussed above; of the rating of provider and likelihood to recommend the practice. Taken together, the two measures are used to describe a patient's loyalty to the practice referred to as the patient loyalty index.

Using Cronbach's alpha, which is a measure of how closely related a set of items are as a group, the reliability estimate for the two primary outcomes was calculated. The results support that the two items are measuring the same construct and therefore appropriate to combine into a singular index. In addition, the patient loyalty index is correlated with all other individual survey items to determine the items' impact on, or correlation with, the two primary outcomes.

To investigate which patient experience survey items predict the patient loyalty index, a Spearman's Rho correlation (a non-parametric test used to measure the strength of association between two variables) was used to examine ordinal by interval associations. The relationship between the correlation and the average ratings for the metrics was examined using a scatter diagram (FIG. 5). Principal drivers of patient loyalty appear in the upper right quadrant. These survey items have been found to have a high correlation with the patient loyalty index such that a high score on these surveys items highly correlates with provider satisfaction ratings and likelihood to recommend the practice (patient loyalty index).

In the example shown, the principal drivers of patient loyalty are: provider explained the plan for follow-up care; provider spent enough time with you; provider listened carefully to you; provider involved you in decisions about your treatment plan; provider discussed treatment options, including the possible risks and benefits; provider knew the important information about your medical history; provider showed respect for what you had to say; provider explained things in a way that was easy to understand; and provider explained the reasons for prescribing medications

In the example shown, the key driver analysis found the greatest opportunities are explained alternative medication/treatment and explained possible side effects.

These appear in the upper left quadrant and suggest that these items have a high correlation with patient loyalty but a low average score. Improvement on these key items would necessarily improve the patient loyalty index.

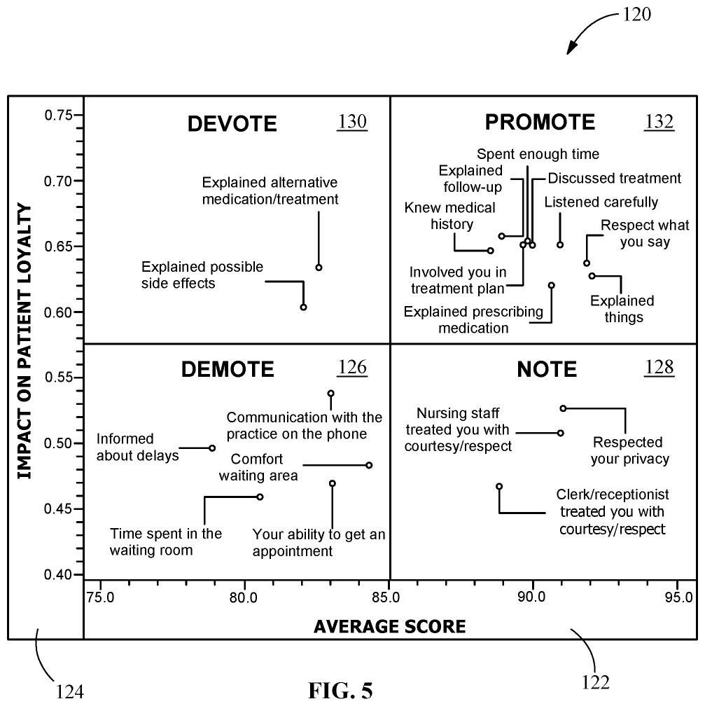

Practices must understand key drivers of business outcomes to help focus improvement initiatives. In order to do so, a key driver analysis must be performed. The key driver analysis examines the statistical relationships between potential drivers and desired outcomes. The key driver matrix (FIG. 4) provides a narrative interpretation of the key driver analysis.

Each quadrant in the key driver matrix includes descriptions that provide the user an explanation of how to use and act on (or not act on) each quadrant's data.

In the illustrative embodiment shown in FIG. 4, the key driver matrix 120 plot the average score 122 along an x-axis and the impact on patient loyalty 124 along a y-axis. Items which are plotted in the lower left quadrant 126 have the lowest priority, and the lowest impact on patient loyalty and over-investment to resolve should be avoided. Items which are plotted in the lower right quadrant 128 have low impact on patient loyalty but may be useful in attracting new patients/customers. These items may be leveraged in marketing and sales. Items which are plotted in the upper left quadrant 130 have strategic opportunities and are key drivers in success to increase patient loyalty. Items which are plotted in the upper right quadrant 132 indicate strengths in patient loyalty and are key drivers in the acquisition and retention of patients/customers. These items may be leveraged in marketing and sales.

FIG. 5 represents an illustrative practice and how various items were scored and plotted. In quadrant 126, items for the illustrative practice included: time spent in the waiting room; ability to get an appointment; information regarding delays; comfort of waiting area; and communication with the practice on the phone. In quadrant 128, items for the illustrative practice included: treatment by receptionist; treatment by the nursing staff; and privacy issues. In quadrant 130, items for the illustrative practice included: explanation of possible side effect; and explanation of alternative medication/treatment. In quadrant 130, items for the illustrative practice included: explanation of medication; general explanation related to treatment; patient involvement in treatment plan; ability of doctor to listen and respect patient; doctor knowing the medical history of the patient; full discussion of treatment; explaining follow-ups; and time spent with patient.

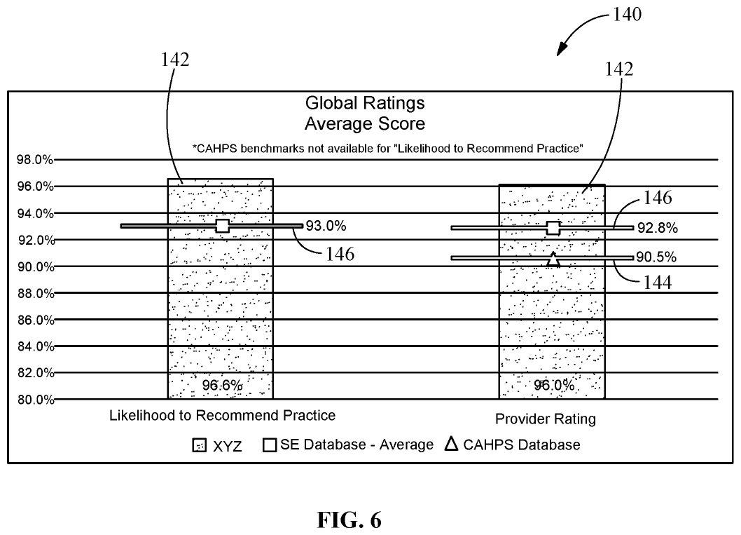

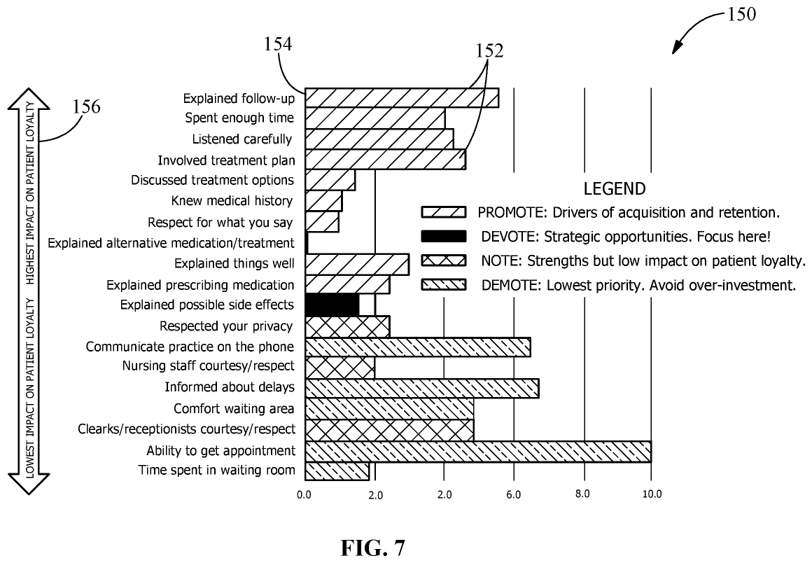

The results of the key driver analysis are presented to the practice group in a visual form 140, for example, as shown in FIG. 6. In the illustrative embodiment shown, the results of the practice group 142 are shown relative to the Comparative Database 144 and the SE Healthcare Database 146.

Referring to FIG. 7, the drivers of patient loyalty shown in FIG. 5, are shown using a gap analysis model 150 to illustrate the practice group results 252 in comparison to a benchmark 154. In the illustrative embodiment shown, the benchmark is the SE Healthcare Database, and all the practice group results exceed the benchmark. In FIG. 7, the gap analysis model 150 is organized to show the items in descending order from the highest impact on patient loyalty to the lowest impact on patient loyalty as indicated by arrow 156. However, the gap analysis model 150 may be organized and displayed using different criteria.

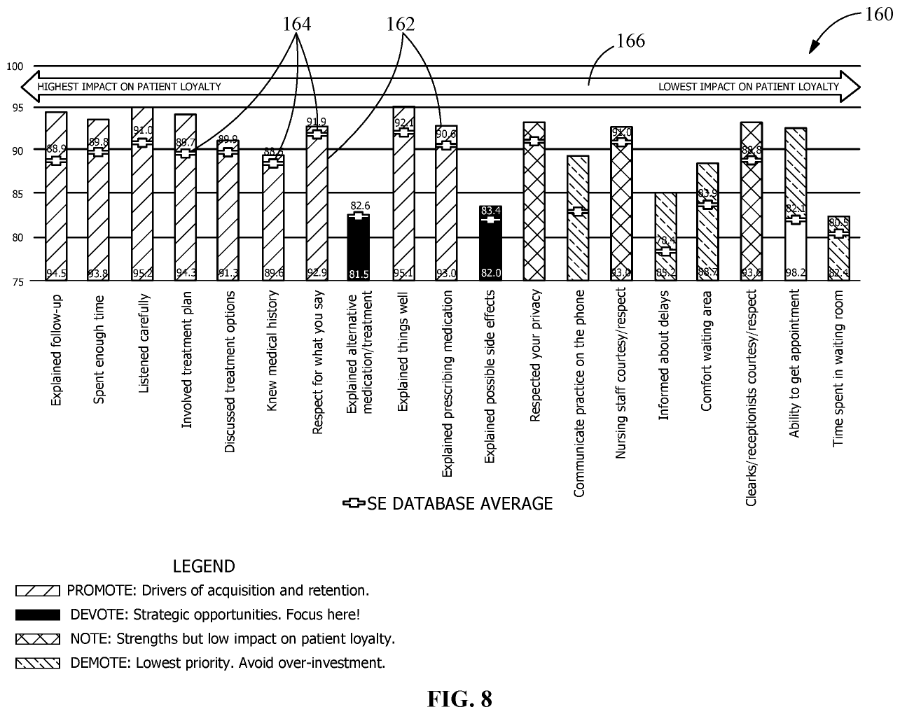

FIG. 8 illustrate another alternate visual or gap analysis model 160 to illustrate the practice group results 162 in comparison to a benchmark 164. In the illustrative embodiment shown, the benchmark is the SE Healthcare Database. In FIG. 8, the model 160 is organized to show the items with the highest impact on patient loyalty on the left to the lowest impact on patient loyalty on the right as indicated by arrow 166.

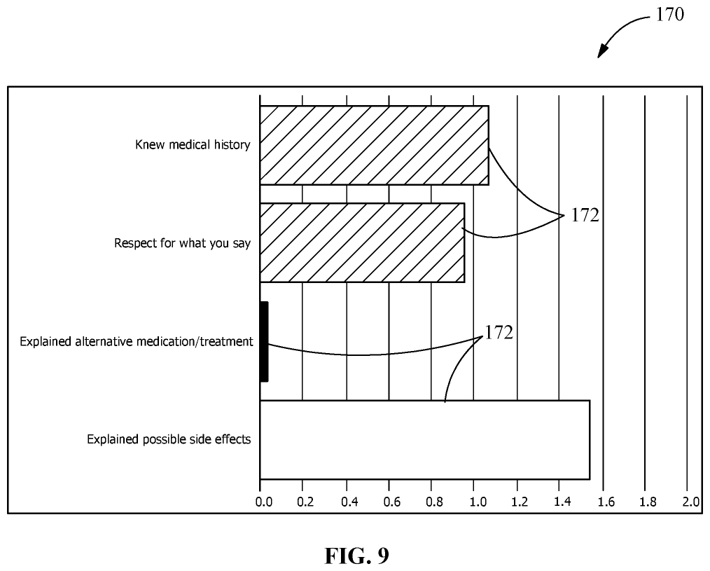

FIG. 9 illustrates a graphic representation 170 of several selected drivers 172. The drivers 172 are selected based on the performance improvement opportunities which may have the greatest impact on the rating of the practice of group.

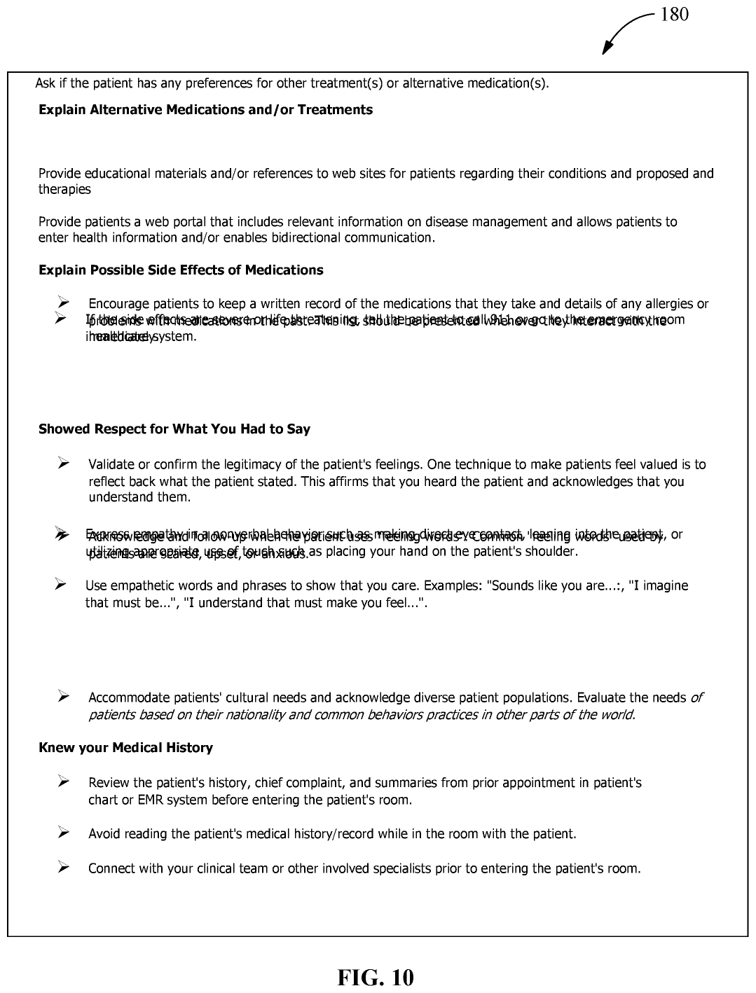

FIG. 10 represents an example of prioritized tips 180 to improve the patient experience. The prioritized tips 180 are generated based on the analysis of the key drivers.

Practices can use this information to align their own patient experience data and identify where they have strengths and weaknesses and most importantly, where to focus resources for improvement.

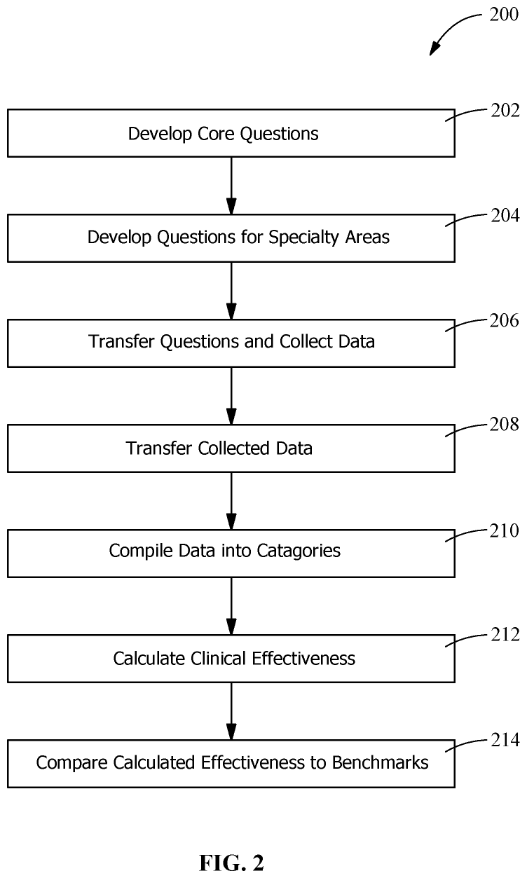

As best shown in FIG. 2, a healthcare management system 200 of determining and enhancing the clinical effectiveness and a method of determining and enhancing the clinical effectiveness with a healthcare practice or group includes the steps of: develop core questions 202 to be answered by physicians or practice groups regarding clinical effectiveness and storing, for example, but not limited to, on a second computer or server; develop questions for various specialty areas 204 of practice to be answered by physicians or practice groups regarding clinical effectiveness and storing, for example, but not limited to, on the second computer or server; transferring the questions 206 to, for example, but not limited to, a first computer or server and collecting data from physicians or practice groups on the first computer or server; transferring the collected data 208, for example, but not limited to, from the first computer or server to the second computer or server; compiling the data 210 into meaningful categories, for example, but not limited to, on the second computer or server; responsive to the step of compiling, calculating clinical effectiveness score 212 for the practice group for each category, for example, but not limited to, on the second computer or server; and responsive to the step of calculating, comparing 214, for example, but not limited to, on the second computer or server, the calculated clinical effectiveness score against national benchmarks.

The clinical effectiveness questions are developed by subject matter experts to solicit information regarding key drivers of the clinical effectiveness of the practice or group. The clinical effectiveness questions include core questions which are applicable to all practices and groups. The clinical effectiveness questions also include subject matter or specialty practice questions which are applicable to the respective specialty practice. Appropriate multiple choice answers are developed for each question. The questions and answers reside on a first computer or server.

One or more physicians of a practice group answers the questions. The answers are sent from the first computer or server to a second computer or server.

The data is gathered, compiled, analyzed and summarized by the second computer or server to generate a clinical effectiveness assessments. The data is compared to benchmarks, such as, but not limited to SE Healthcare's national database. The benchmarks from the SE Healthcare's national database are continually updated to ensure that the benchmarks are based on the most current data available.

The clinical effectiveness assessments include evidenced-based medicine and/or clinical practice guidelines along with measures of efficiency; health and burnout; patient experience/engagement; and patient safety. The clinical effectiveness assessments assess clinical and operational practices beyond the scope of many quality/regulatory reporting programs. The content is thoroughly vetted by a team of internal and external subject matter experts.

Clinical effectiveness assessments include a “just-in-time” learning approach, allowing for immediate identification of knowledge or practice gaps. Where gaps exist, providers use clinical effectiveness assessments for educational and practice-based quality improvement initiatives.

Clinical effectiveness assessment data describes where a practice lies on the “quality continuum” relative to peers. Providers who obtain high clinical effectiveness assessment scores are practicing safe, high quality and efficient medicine.

Each clinical effectiveness assessment item is categorized into one of five composite categories. Content related to specialty-specific evidenced-based medicine and/or clinical practice guidelines is categorized as clinical quality. Other composites include efficiency; health and burnout; patient experience/engagement and patient safety. Items are individually weighted for impact on each of the five composite categories. For example, developing an individual written plan of care with the patient that reflects the patient's preferences, values, capabilities, and goals is weighted higher for impact on patient experience/engagement and lower on health and burnout.

The Institute of Medicine defines health care quality as “[t]he degree to which health care services for individuals and populations increase the likelihood of desired health outcomes and are consistent with current professional knowledge”. The clinical quality category is comprised of developed specialty-specific content based on recommendations or statements set forth by medical associations, professional societies, government/regulatory agencies or peer-reviewed scientific research.

Efficiency measures whether healthcare resources are being used to get the best value for money. Efficiency is the relation between resource inputs (costs, in the form of labor, capital, or equipment) and either intermediate outputs (numbers treated, waiting time, etc.) or final health outcomes (lives saved, life years gained, quality adjusted life years) with an ideal focus on final health outcomes.

Burnout is defined as “ . . . a syndrome characterized by a loss of enthusiasm for work (emotional exhaustion), feeling of cynicism (depersonalization), and a low sense of personal accomplishment”. The effects of burnout include increased medical errors; riskier prescribing practices; and lower patient compliance with chronic disease management plans. Burned out physicians report a lack of empathy and are more likely to leave the profession.

The patient experience, simply stated, is a measure of whether something that should happen in a health care setting (such as clear communication with a provider) actually happened or how often it happened.

Patient engagement is a concept that combines a patient's knowledge, skills, ability and willingness to manage his own health and care with interventions designed to increase activation and promote positive patient behavior.

Patient safety as defined by the Institute of Medicine as “[t]he prevention of harm to patients.” Emphasis is placed on the system of care delivery that (a) prevents errors; (b) learns from the errors that do occur; and (c) is built on a culture of safety that involves health care professionals, organizations, and patients.

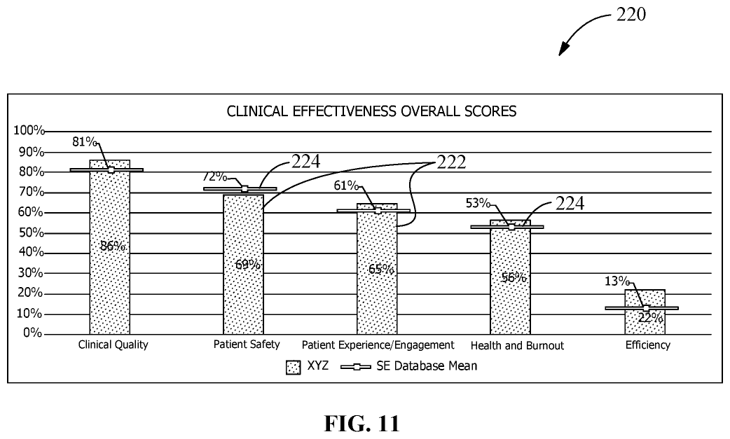

The results of the composite categories of the clinical effectiveness of the practice or group are presented in a visual form, for example, as shown in FIG. 11. In the illustrative embodiment shown, a visual or gap analysis model 220 illustrates the practice group results 222 in comparison to a benchmark 224. In the illustrative embodiment shown, the benchmark is the SE Healthcare Database.

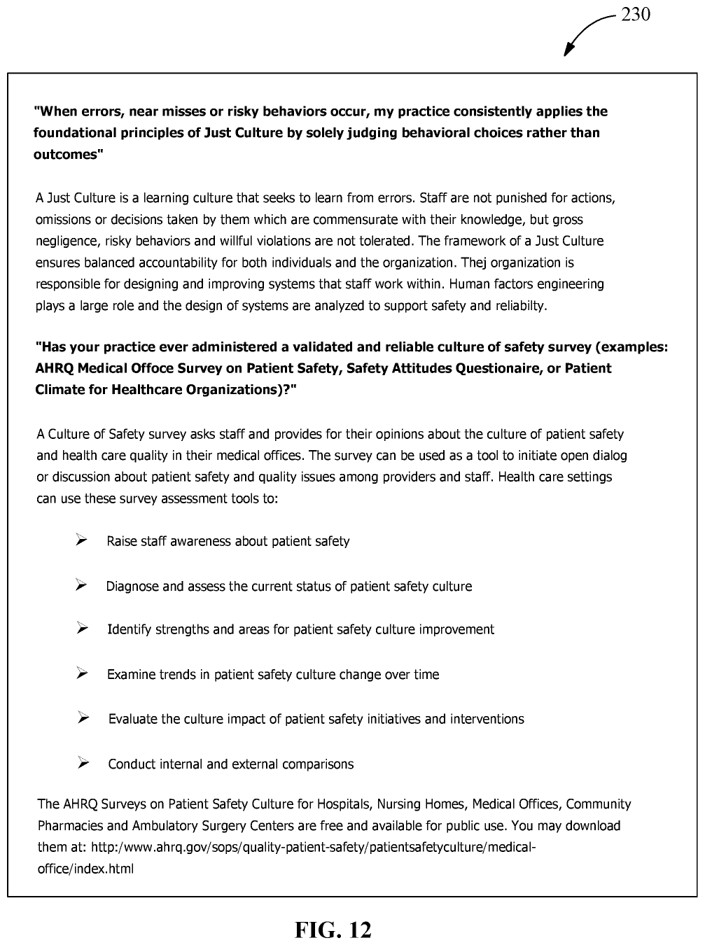

FIG. 12 represents an example of prioritized tips 230 to improve the clinical effectiveness. The prioritized tips are generated based on the analysis of the composite categories of the clinical effectiveness.

Practices can use this information to align their own clinical effectiveness data and identify where they have strengths and weaknesses and most importantly, where to focus resources for improvement.

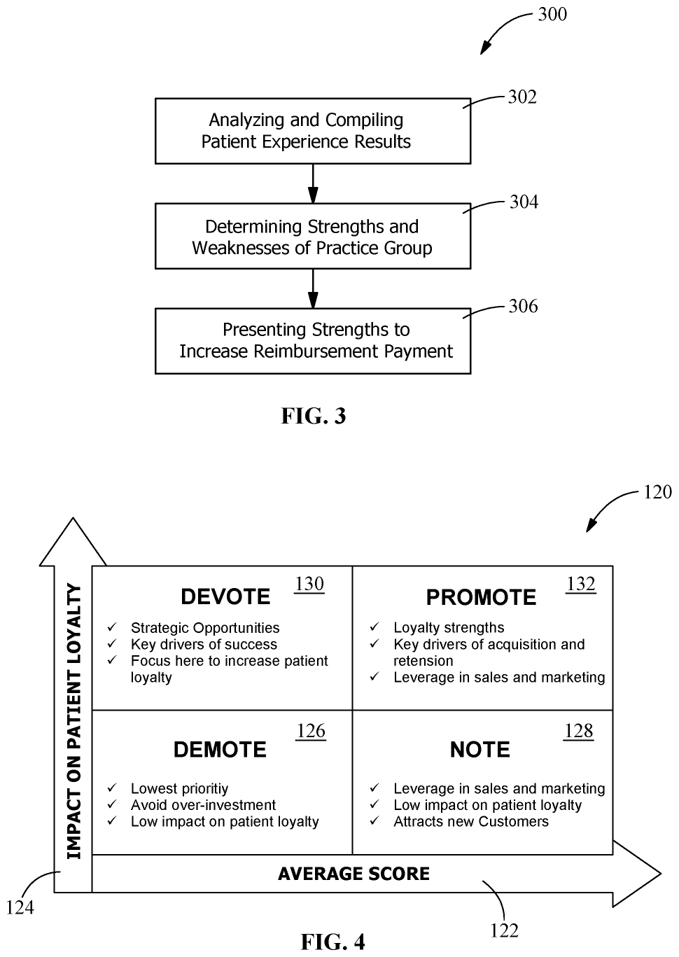

As best shown in FIG. 3, the healthcare management system 300 for determining and enhancing the reimbursement effectiveness and a method of determining and enhancing the reimbursement effectiveness of the practice or group includes: analyzing and compiling 302, for example, but not limited to, on a first computer or server, the patient experience results, as described herein, and the clinical effectiveness results, as described here; responsive to the step of analyzing and compiling, determining the strengths of the practice of group 304; and presenting the strengths of the practice of group to the insurance provider 306 to demonstrate risk mitigation and to increase the reimbursement payment or percentage that the practice of group receives from the insurance provider.

The reimbursement enhancement or effectiveness of the practice can be enhanced by utilizing the results of the patient experience and clinical effectiveness described above. A computer or server analyzes and compiles the patient experience results and the clinical effectiveness results to determine the strengths of the practice of group. The strengths of the practice or group are then presented to the insurance provider to demonstrate risk mitigation and to increase the reimbursement payment or percentage that the practice of group receives from the insurance provider.

While the invention has been described with reference to a preferred embodiment, it will be understood by those skilled in the art that various changes may be made, and equivalents may be substituted for elements thereof without departing from the spirit and scope of the invention as defined in the accompanying claims. In particular, it will be clear to those skilled in the art that the present invention may be embodied in other specific forms, structures, arrangements, proportions, sizes, and with other elements, materials and components, without departing from the spirit or essential characteristics thereof. One skilled in the art will appreciate that the invention may be used with many modifications of structure, arrangement, proportions, sizes, materials and components and otherwise used in the practice of the invention, which are particularly adapted to specific environments and operative requirements without departing from the principles of the present invention. The presently disclosed embodiments are therefore to be considered in all respects as illustrative and not restrictive, the scope of the invention being defined by the appended claims, and not limited to the foregoing description or embodiments.

1. A method of determining and enhancing a patient experience with a healthcare practice or group, the method comprising:
collecting data from a patient on a first computer;
transferring the collected data from the first computer to a second computer or server;
compiling, by the second computer or server, the data into defined categories on the second computer or server;
comparing, on the second computer or server the compiled data against benchmarks;
determining and ranking key drivers of patient loyalty on the second computer or server; and
determining the healthcare practice or group performance on the second computer or server.

2. The method as recited in claim 1, further comprising presenting the ranking of the key drivers in visual form.

3. The method as recited in claim 1, further comprising using a gap analysis model to illustrate the key drivers of patient loyalty in comparison to a benchmark.

4. The method as recited in claim 1, further comprising identifying the healthcare practice or group performance strengths, weaknesses and where to focus resources for improvement.

5. The method as recited in claim 1, wherein the key drivers of patient loyalty selected from the group consisting essentially of: provider explained the plan for follow-up care; provider spent enough time with you; provider listened carefully to you; provider involved you in decisions about your treatment plan; provider discussed treatment options, including the possible risks and benefits; provider knew the important information about your medical history; provider showed respect for what you had to say; provider explained things in a way that was easy to understand; and provider explained the reasons for prescribing medications.

6. The method as recited in claim 5, further comprising mapping the key drivers of patient loyalty in a key driver matrix to identify key drivers on which improvements would improve the patient loyalty.

7. The method as recited in claim 5, further comprising examining statistical relationships between potential drivers and desired outcomes.

8. A method of determining and enhancing a clinical effectiveness of a healthcare practice or group, the method comprising:
developing core questions to be answered by physicians or the healthcare practice or group regarding clinical effectiveness and storing the core questions on a second computer or server;
developing specialty questions for various specialty areas of healthcare practice or group to be answered by the physicians or healthcare practice or group regarding clinical effectiveness and storing the specialty questions on the second computer or server;
transferring the core questions and relevant specialty questions to a first computer;
collecting data from physicians or healthcare practice or group on the first computer;
transferring the collected data from the first computer to the second computer or server;
compiling the collected data into categories on the second computer or server;
calculating a clinical effectiveness score for the healthcare practice or group for each category on the second computer or server; and
comparing on the second computer or server, the calculated clinical effectiveness score against benchmarks.

9. The method as recited in claim 8, wherein the core questions are applicable to all practices and groups.

10. The method as recited in claim 8, wherein specialty questions include subject matter which is applicable to the respective specialty practice.

11. The method as recited in claim 8, further comprising continually updating databases of the benchmarks to ensure that the benchmarks are based on the most current data available.

12. The method as recited in claim 8, wherein the collected data includes measures of efficiency; health and burnout; patient experience/engagement; and patient safety.

13. The method as recited in claim 12, wherein data is individually weighted for impact on each of the measures.

14. A method of determining and enhancing the reimbursement effectiveness of a healthcare practice or group, the method comprising:
determining a patient experience with a healthcare practice or group, the method of determining a patient experience comprising:
collecting data from a patient;
compiling the data into defined categories;
comparing the compiled data against first benchmarks;
determining and ranking key drivers; and
determining the healthcare practice or group performance;
determining a clinical effectiveness of a healthcare practice or group, the method of determining a patient experience comprising:
developing core questions to be answered by physicians or the healthcare practice or group regarding clinical effectiveness;
developing specialty questions for various specialty areas of healthcare practice or group to be answered by the physicians or healthcare practice or group regarding clinical effectiveness;
collecting data is response to the core questions and specialty questions from physicians or healthcare practice or group;
compiling the collected data into categories;
calculating a clinical effectiveness score for the healthcare practice or group for each category; and
comparing the calculated clinical effectiveness score against second benchmarks;
analyzing and compiling the patient experience results and the clinical effectiveness results;
determining the strengths of the healthcare practice or group; and
presenting the strengths of the healthcare practice or group to an insurance provider to demonstrate risk mitigation and to increase the reimbursement payment or percentage that the healthcare practice or group receives from the insurance provider.

15. The method as recited in claim 14, further comprising using a gap analysis model to illustrate the key drivers of patient loyalty in comparison to a benchmark.

16. The method as recited in claim 14, further comprising mapping the key drivers of patient loyalty in a key driver matrix to identify key drivers on which improvements would improve the patient loyalty.

17. The method as recited in claim 14, further comprising examining statistical relationships between potential drivers and desired outcomes.

18. The method as recited in claim 14, further comprising continually updating databases of the second benchmarks to ensure that the second benchmarks are based on the most current data available.

19. The method as recited in claim 14, wherein the collected data includes measures of efficiency; health and burnout; patient experience/engagement; and patient safety.

20. The method as recited in claim 14, wherein data is individually weighted for impact on each of the measures.
